(19) United States
(12) Patent Application Publication
(10) Pub. No.: US 2012/0287403 A1
(43) Pub. Date: Nov. 15, 2012

(54) ILLUMINATION METHOD AND APPARATUS FOR USING A MULTIMODE FIBER OPTIC TO PROVIDE VARIABLE ILLUMINATION SCHEMES

(76) Inventor: Douglas E. Music, Wildwood, MO (US)

(21) Appl. No.: 13/104,462

(22) Filed: May 10, 2011

(51) Int. Cl. A61B 3/10

(52) U.S. Cl. 351/221

(57) ABSTRACT

An ophthalmic illumination system comprising a multimode fiber optic having an end a first laser source, and the system configured to project light into the end at a plurality of different angles. An ophthalmic illumination system comprising a multimode fiber optic having an end, a first light source adapted to produce light of a first color, a second light source adapted to produce light of a second color, and a beam combiner configured and arranged to receive and combine the light from the first light source and the light from the second light source to form a combination of light. The beam combiner is configured and arranged to project the combination of light into the end. The system is adapted to vary at least one of the amount of the light of the first color and the amount of the light of the second color projected into the end, such that the combination of light has a selectable color.

### FIELD OF INVENTION

The present invention relates to illumination methods and apparatus for performing ophthalmic surgical procedures, and more particularly to illumination methods and apparatus for performing ophthalmic surgical procedures that facilitate providing variable illumination schemes.

### BACKGROUND OF THE INVENTION

Illumination used for performing intraocular surgical procedures is significant to achieving desirable results. Illumination color and pattern shape are relevant parameters to providing appropriate illumination.

Conventionally, multiple hand pieces and/or light sources have been necessary to provide appropriate illumination for the multitude of different procedures that a surgical staff is called upon to perform.

### SUMMARY

Aspects of the present invention are directed to an ophthalmic illumination system, comprising a multimode fiber optic having an end, and a first laser source, the system configured to project light into the end at a plurality of different angles.

In some embodiments, the system further comprises a mount having at least one of the fiber optic and the first laser source coupled thereto, the mount configured to permit relative movement between the fiber optic and the first laser source such that the first laser source can project light into the end at a plurality of different angles.

In some embodiments, the system further comprises a second laser source positioned to project light into the end at a fixed angle that is determined independently of the plurality of different angles. The second laser source may provide the light along an optical axis of the fiber optic. The mount may be rotatable relative to the end. The mount may be continuously rotatable relative to the end. In some embodiments, the system comprises a handpiece in which a second end of the multimode fiber optic is disposed.

Another aspect of the invention is directed to a method of illuminating an eye, comprising projecting laser light into a multimode fiber optic having an end, the light projected into the end at a plurality of different angles in the manners described herein.

Other aspects of the invention are directed to an ophthalmic illumination system, comprising a multimode fiber optic having an end, a first light source adapted to produce light of a first color, a second light source adapted to produce light of a second color, and a beam combiner configured and arranged to receive and combine the light from the first light source and the light from the second light source to form a combination of light, and the beam combiner configured and arranged to project the combination of light into the end, the system being adapted to permit manual variation of at least one of the amount of the light of the first color and the amount of the light of the second color projected into the end, such that the combination of light has a selectable color.

In some embodiments, the first light source comprises a laser. In some embodiments, the first light source comprises an LED.

The system may be adapted to permit manual variation of at least one of the amount of the light of the first color and the amount of the light of the second color projected into the end.

In some embodiments, the system comprises a processor, wherein the processor is adapted to vary at least one of the amount of the light of the first color and the amount of the light of the second color projected into the end.

The system may be configured to project the combination of light into the end at a plurality of different angles.

In some embodiments, the system further comprises a third light source adapted to produce light of a third color, a fourth light source adapted to produce light of a fourth color; and a second beam combiner configured and arranged to receive and combine the light from the third light source and the light from the fourth light source to form a second combination of light, and the second beam combiner configured and arranged to project the second combination of light into the end.

The system may be adapted to vary at least one of the amount of the light of the third color and the amount of the light of the fourth color projected into the end, whereby the second combination of light has a selectable color. In some embodiments, the system is configured to project the second combination into the end at a fixed angle.

A further aspect of the invention is directed to a method of illuminating an eye, comprising providing a multimode fiber having an end, projecting light of a first color and light of a second color, and combining the light of the first color and the light of the second color to form a combination of light, and projecting the combination of light into the end, and varying at least one of the amount of the light of the first color and the amount of the light of the second color projected into the end, whereby the combination of light has a selectable color.

### BRIEF DESCRIPTION OF THE DRAWINGS

Illustrative, non-limiting embodiments of the present invention will be described by way of example with reference to the accompanying drawings, in which the same reference number is used to designate the same or similar components in different figures, and in which:

FIG. 1A is a schematic illustration of an example of an ophthalmic illumination system according to aspects of the present invention;

FIG. 1B is a view of an illumination pattern at plane P in FIG. 1A;

FIG. 2 is a schematic illustration of an example of an ophthalmic illumination system according to aspects of the present invention;

FIG. 3 is a schematic illustration of an example of an ophthalmic illumination system according to aspects of the present invention; and

FIGS. 4A-4B are schematic illustrations of examples of ophthalmic illumination systems according to aspects of the present invention in which steering optics are used to project light into a fiber at a plurality of angles.

### DETAILED DESCRIPTION

An aspect of the present invention is directed to an ophthalmic illumination system comprising light from one or more laser sources that are directed through a multimode fiber at various input angles. It will be appreciated that laser sources (e.g., gas lasers or solid state lasers) provide light in a relatively controlled manner due to the relatively high directionality of laser sources. The Applicant has found that projection of such light (i.e., a beam) through a multimode fiber optic results in a useful illumination pattern that is controllable by altering the input angle of the light into the fiber optic. By appropriately controlling movement of the laser source (or a direction of light from the laser source) relative to the fiber, light outputs from the fiber having different light patterns can be achieved, where the various light outputs are useful during a given procedure or during different procedures. According to aspects of the invention, the first laser source is disposed on a mount moveable relative to the fiber optic such that the laser source can project light into an end of the fiber at a plurality of different angles whereby movement of the mount alters the pattern of light that emerges from the fiber optic after the light has propagated through the fiber (i.e., via multiple total internal reflections).

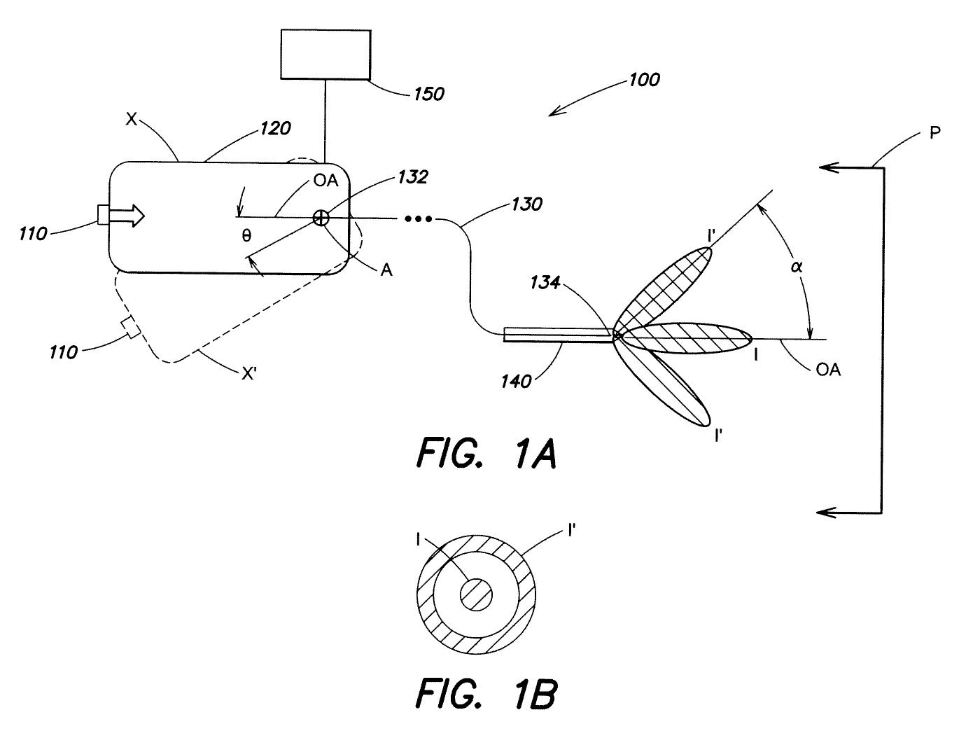

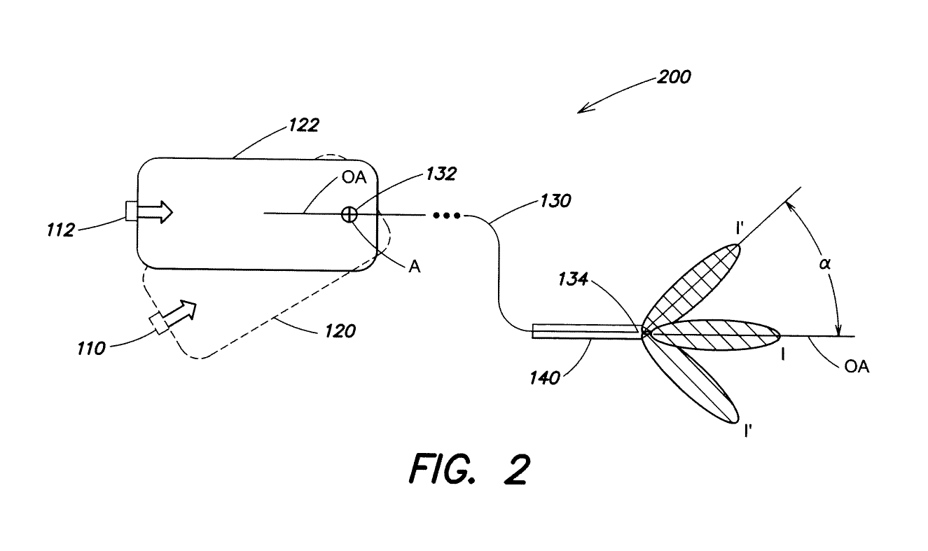

FIG. 1A is a schematic illustration of an example of an ophthalmic illumination system 100 according to aspects of the present invention. Illumination system 100 comprises a first laser source 110, a mount 120 having laser source 110 coupled thereto, fiber optic 130, and a hand piece 140.

First laser source 110 can be any suitable laser providing a light output that by itself or in combination with another laser source provides light of a suitable wavelength or spectrum of wavelengths. Typically the light is within the visible spectrum. While the laser source can be a solid state laser, a liquid laser, or gas laser or other suitable laser configuration, in some embodiments, laser diodes are advantageous due in part to their longevity, durability and relatively low cost.

Multimode fiber optic 130 may be any suitable fiber optic for propagating light from laser source 110. One of ordinary skill in the art will understand that a suitable fiber will have suitable mechanical, thermal as well as other properties, in addition to being capable of providing suitable illumination patterns. Light from laser source 110 may be coupled into fiber optic 130 using any suitable technique, such as using a conventional end fire coupling technique into end 132. The light propagates to a second end 134 of the fiber optic in a conventional manner via multiple total internal reflections. Typically, the light emerges from the second end within or proximate the distal end of a hand piece 140.

FIG. 1B is a view of the illumination pattern at plane P in FIG. 1A. As illustrated in FIGS. 1A and 1B, the result of propagating highly directional light though a multimode fiber optic is to achieve a selected illumination pattern and a decrease in coherence; however the pattern is dependent on the angle θ at which the light enters end 132. If the light is propagated along an optical axis OA of fiber optic 130 (θ=0), as indicated by a position X, the emergent light will remain along optical axis OA upon exit from second end 134 to form a pattern I at plane P. If the fiber is moved to a second position X′, where the light is projected at an angle relative to the end of the fiber (θ≠0), the emergent light will propagate in a conical shape to form pattern I′ at plane P. The angle α at which the light emerges from end 134 is determined by angle θ at which the light is input relative to optical axis OA as well as the wavelength of the light, the diameter of the fiber, index of refraction of the fiber and other properties. Significantly, the pattern of the illumination that emerges from the fiber optic is selectable by adjusting angle θ at which light is input at end 132.

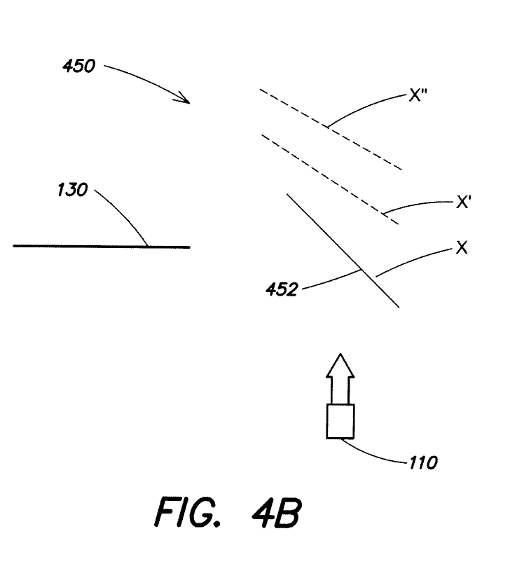

Although relative angular positioning between the end of the fiber optic and the light input is pertinent, for purposes of illustration and discussion, the embodiment in FIG. 1A is illustrated with a mount 120 that has first laser source 110 coupled thereto, the mount being moveable relative to fiber optic end 132 such that first laser source 110 can project light into end 132 at a plurality of different angles θ. Embodiments of the invention include a mount having at least one of the fiber optic and the laser source coupled thereto, the mount configured to permit relative movement between the fiber optic and the first laser source such that the first laser source can project light into end 132 at a plurality of different angles. Although the embodiments described above have the laser source and/or fiber optic coupled to the mount to project the light into the end at different angles, as described below with reference to FIGS. 4A-4B, in some embodiments, the source and fiber optic may remain stationary and one or more steering optics (e.g., one or more mirrors; not shown) are configured to direct the light into end 132 at different angles. Mount 120 may have any configuration suitable for maintaining laser source 110, fiber optic 130 and/or steering optics, and thereby permitting angular positioning of the light input relative to the optical axis OA.

Also, although mount 120 is illustrated as permitting only angular positioning of fiber optic 130 about an axis A, it will be appreciated that mount 120 may provide angular and/or translational movement provided it permits variation in the input angle θ.

Laser source 110, fiber optic 130 and/or steering optics may be moveable in discrete amounts or may be continuously moveable to achieve variations in input angles θ. For example, mount 120 may be moveable in discrete amounts to achieve multiple positions X and X′ (and corresponding angles θ) or may be continuously moveable between positions X and X′. Movement may be manual or under the control of a processor 150. Processor 150 may operate in response to operator inputs (e.g., using a keyboard or a graphical user interface) or may be preprogrammed.

Any suitable technique for maintaining and positioning fiber end 134 to direct emergent light within an eye may be used. Hand piece 140 which is sized and shaped to enter an incision in an eye (e.g., typically having an overall diameter equal to or less than 3 mm) represents one possible technique. Hand piece 140 may have any suitable configuration for facilitating manipulation of end 134.

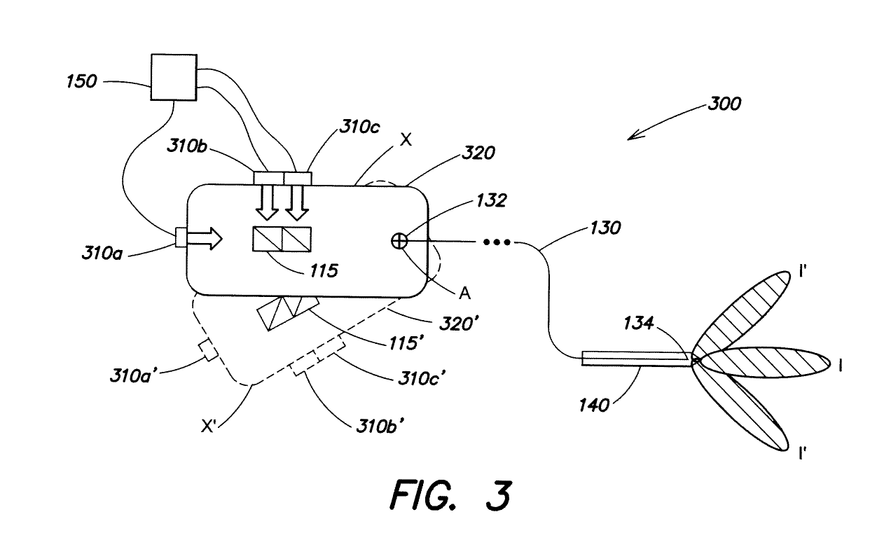

FIG. 2 is a schematic illustration of another example of an ophthalmic illumination system 200 according to aspects of the present invention. System 200 is configured the same as system 100 described above with reference to FIG. 1 including a laser source 110; however system 200 further comprises a second laser source 112 positioned to project light into end 132 at a fixed angle. In the illustrated embodiment, second laser source 112 is coupled to a second mount 122; however, such a configuration is not necessary and any suitable structure for positioning second laser source 112 relative to fiber optic 130 at a fixed angle may be used. Although second source 112 is positioned at a fixed angle relative to end 132, it may be movable such that the fixed angle can be altered (e.g., by moving mount 122) or the source may be immovable relative to end 132.

In some embodiments, the fixed angle is along the optical axis OA of the fiber optic. It will be appreciated that, if light is projected along the optical axis at end 132 (θ=0) it will form a pattern along the optical axis (α=0) at the second end 134 of the fiber (e.g., as illustrated by pattern I).

As described above with reference to FIG. 1, movement of light relative to end 132 (e.g., by movement of mount 120) will allow adjustability of light pattern I′. The overall illumination pattern is formed by light patterns I and I′. Such a configuration will provide light on-axis and will also provide light in the periphery. Patterns I and I′ may be provided at an adjustable angle α, by adjusting input angle θ of laser sources 110 and 112.

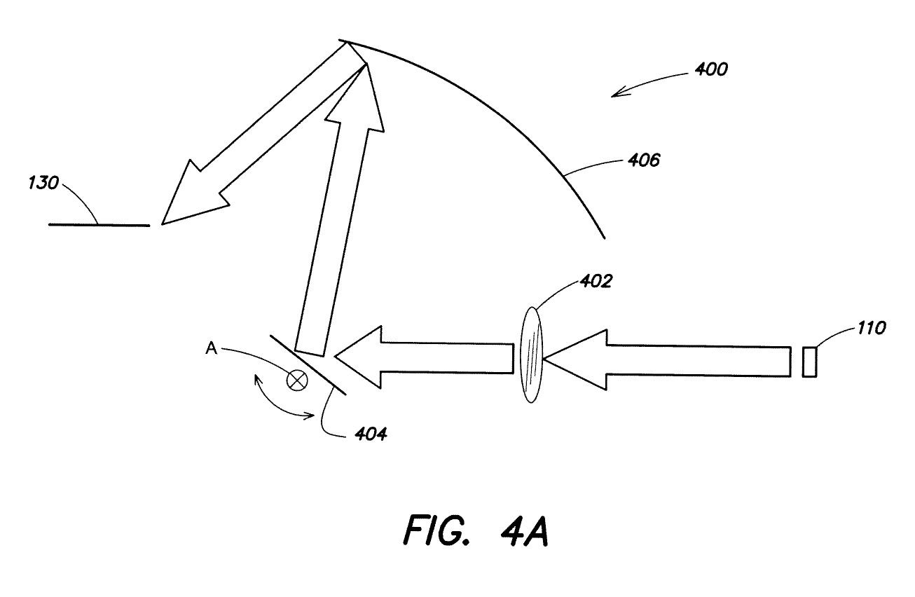

FIG. 3 is a schematic illustration of an example of an ophthalmic illumination system 300 according to aspects of the present invention. Ophthalmic illumination system 300 comprises a multimode fiber optic 130, a hand piece 140, and a light subsystem comprising a beam combiner 115, a first light source 310a adapted to produce light of a first color, a second light source 130b adapted to produce light of a second color, and a third light source adapted to produce light of a third color 310c.

Multimode fiber optic 130 has an end 132 and may be configured in any manner as described above with reference to FIG. 1.

First light source 310a is adapted to produce light of a first color. Second light source 310b is adapted to produce light of a second color that is different than the first color. Optionally, a third 310c or more additional light sources may also be present that produce light of different colors than light sources 310a and 310b. One or more of the light sources 310a—310c may be a laser light source or an LED light source.

Beam combiner 115 is configured and arranged to receive and combine the light from first light source 310a and the light from the second light source 310b to form a combination of light. The beam combiner is configured and arranged to project the combination of light into end 132. The beam combiner can be of any suitable configuration for combining light from two or more light sources 310a—310c. For example, the combiner may comprise a prism beam combiner as shown in FIG. 3.

System 300 is adapted to vary at least one of the amount of the light of the first color and the amount of the light of the second color that is projected into end 132. Any suitable technique for altering an amount of light from the light sources may be used. For example, the amount of light emitted by the light sources can be altered manually or under the control of processor 150 having an input device such as a keyboard or a graphical user interface to select amounts of light emitted by the light sources. Alternatively or additionally, to alter an amount of light, one or more attenuation filters (not shown) may be suitably located in a light path of one or more of light source 310a-310c and can be manipulated manually or under the control of a processor 150. It will be appreciated that, by altering the amount of light, the color of the combination of light is selectable.

Some embodiments of system 300 are configured such that the combination of light can be projected into end 132 at a plurality of different angles as was described above with reference to FIG. 1. For example, system 300 may comprise a mount 320 having at least one of the fiber optic 130, combiner 115 or steering optics (e.g., a mirror (not shown)) coupled thereto, the mount configured to permit relative movement between the fiber optic and the combination of light.

Some embodiments of system 300 may comprise a second light subsystem comprising a light source 310a′, a second light source 310b′, one or more additional light sources 310c′, and a combiner 115′. The second light subsystem may be configured as light sources 310a—310c and combiner 115 described above, including producing light having colors as described above. Additionally, light sources 310a′-310c′ and combiner 115′ may be configured and arranged to project light into end 132 at a fixed angle as described above with reference to FIG. 2.

FIGS. 4A-4B are schematic, illustrations of examples of ophthalmic illumination systems 400 and 450 according to aspects of the present invention in which steering optics are used to project light into fiber 130 at a plurality of angles, while the laser source 110 and fiber 130 remain stationary. In FIG. 4A, light from laser source 110 is projected through a lens 402 having a relatively long focal length. A planar mirror 404 that is rotatable about an axis A causes the light to project onto various portions of a suitably curved mirror 406. As mirror 404 is rotated, light enters fiber 130 at various angles. It will also be appreciated that the light can be directed using a non-moving steering optic such as an acousto-optical component. As shown in FIG. 4B, in some embodiments, a single planar mirror 452 can be suitably translated and rotated through a plurality of positions X, X′, X″ using gears and other guidance apparatus (not shown) to project light into fiber 130 at a plurality of angles, while the laser source 110 and fiber 130 remain stationary.

Having thus described the inventive concepts and a number of exemplary embodiments, it will be apparent to those skilled in the art that the invention may be implemented in various ways, and that modifications and improvements will readily occur to such persons. Thus, the embodiments are not intended to be limiting and presented by way of example only. The invention is limited only as required by the following claims and equivalents thereto.

What is claimed is:

1. An ophthalmic illumination system, comprising:
a multimode fiber optic having an end; and
a first laser source, the system configured to project light into the end at a plurality of different angles.

2. The system of claim 1, further comprising a mount having at least one of the fiber optic and the first laser source coupled thereto, the mount configured to permit relative movement between the fiber optic and the first laser source such that the first laser source can project light into the end at a plurality of different angles.

3. The system of claim 1, further comprising a second laser source positioned to project light into the end at a fixed angle that is determined independently of the plurality of different angles.

4. The system of claim 3, wherein the second laser source provides the light along an optical axis of the fiber optic.

5. The system of claim 2, wherein the mount is rotatable relative to the end.

6. The system of claim 5, wherein the mount is continuously rotatable relative to the end.

7. The system of claim 1, further comprising a handpiece in which a second end of the multimode fiber optic is disposed.

8. An ophthalmic illumination system, comprising:
a multimode fiber optic having an end;
a first light source adapted to produce light of a first color;
a second light source adapted to produce light of a second color; and
a beam combiner configured and arranged to receive and combine the light from the first light source and the light from the second light source to form a combination of light, and the beam combiner configured and arranged to project the combination of light into the end,
the system adapted to permit variation of at least one of the amount of the light of the first color and the amount of the light of the second color projected into the end,
whereby the combination of light has a selectable color.

9. The system of claim 8, wherein the first light source comprises a laser.

10. The system of claim 8, wherein the first light source comprises an LED.

11. The system of claim 8, wherein the system is adapted to permit manual variation of at least one of the amount of the light of the first color and the amount of the light of the second color projected into the end.

12. The system of claim 8, further comprising a processor, wherein the processor is adapted to vary at least one of the amount of the light of the first color and the amount of the light of the second color projected into the end.

13. The system of claim 8, wherein the system is configured to project the combination of light into the end at a plurality of different angles.

14. The system of claim 8, further comprising:
a third light source adapted to produce light of a third color;
a fourth light source adapted to produce light of a fourth color; and
a second beam combiner configured and arranged to receive and combine the light from the third light source and the light from the fourth light source to form a second combination of light, and the second beam combiner configured and arranged to project the second combination of light into the end.

15. The system of claim 14, wherein the system is adapted to vary at least one of the amount of the light of the third color and the amount of the light of the fourth color projected into the end, whereby the second combination of light has a selectable color.

16. The system of claim 14, wherein the system is configured to project the second combination into the end at a fixed angle.
